(19) United States
(12) Patent Application Publication
(10) Pub. No.: US 2013/0053680 A1
(43) Pub. Date: Feb. 28, 2013

(54) System and Method for Identification of Medical Device

(71) Applicant: George Frey, Englewood, CO (US)

(72) Inventor: George Frey, Englewood, CO (US)

(21) Appl. No.: 13/593,273

(22) Filed: Aug. 23, 2012

Related U.S. Application Data
(60) Provisional application No. 61/526,352, filed on Aug. 23, 2011.

(51) Int. Cl. A61B 5/055; A61B 6/03; A61B 6/00

(52) U.S. Cl. 600/411; 600/424; 382/128

(57) ABSTRACT

A system and method for rapidly and accurately identifying an implantable medical device is disclosed, said identification including, for example, the manufacturer and model or type of the implantable device, and further assisting the surgeon and other medical professionals in identifying the instruments used to adjust and/or remove the medical device.

### CROSS-REFERENCE TO RELATED APPLICATIONS

The present application claims priority from U.S. Provisional Application No. 61/526,352, filed Aug. 23, 2011, the entire disclosure of which is hereby incorporated by reference.

### FIELD OF INVENTION

The present invention relates generally to a system and method of rapidly identifying a medical implant device and data associated with the device after implantation and apparatus employed to rapidly identify the medical implant.

### BACKGROUND OF THE INVENTION

Medical implants are used in various surgical procedures to support or replace organs, bone, tissue or vessels. Information on the implant in use is sometimes entered in the patient's records in the hospital by medical personnel, but often there is little or no recorded data regarding the manufacturer, model or type of implant a patient has received from a prior surgery. This can create problems for other surgeons and during subsequent medical procedures in or around the area where the unrecorded device has been implanted, or create delays when a surgeon or other medical professional needs to identify, update, service, replace, or augment the implanted device.

With the frequency of recalls initiated by manufacturers and by the United States Food and Drug Association (“FDA”), there is greater emphasis being placed on the ability to identify and track implanted medical devices. A copy of a May 4, 2012 article published in the Denver Post, which is incorporated by reference herein in its entirety, demonstrates the importance of quickly and efficiently identifying a recalled implant: http://www.denverpost.com/nationworld/ci_{—}20544821/fda-lacks-system-trackingmedical-devices-that-malfunction?IADID=Search-www.denverpost.com-www.denverpost.com

One disadvantage of the prior art systems and methods is that the patient's record is archived in the hospital that carried out the implant, and therefore the patient has no information regarding the inserted implant. In an emergency or during a visit to a different doctor or different clinic, or during a follow-up surgery, the patient's record with the information on the implant must first be requested. Sometimes the patient has forgotten whether he is carrying an implant or may be unconscious after an accident, and cannot tell the medical personnel that he is carrying an implant. If it is a metal implant, this can be dangerous for the patient if, for example, he is being subjected to an examination using a magnetic resonance system. Because of these and other disadvantages, it is therefore usually necessary to examine the patient using an imaging process such as X-ray, CT or ultrasound to determine whether he is carrying an implant. However, this imaging procedure provides no detailed information, e.g. regarding the date of the implantation or the serial number of the implant.

In addition to biological and artificial permanent implants, auxiliary medical instruments and temporary implants that are removed after a certain time are often used. Such temporary implants are, for example, screws for fixing bones or attaching other implantable devices to the patient's boney anatomy. There are also a number of surgical instruments that are used when operating on the body of a patient, such as temporary heart pacemaker electrodes, catheters, guidewires, operating clamps, etc. The advantage of rapidly identifying the medical instrument is that it precludes the possibility of surgical instruments and temporary implants being left forgotten in the body of the patient during an operation or that have failed to have been removed in a timely and safe manner.

Surgeons now have the ability to readily convert, for example, x-ray data, magnetic resonance imaging (MRI) data or computed tomography (CT) data into usable image data for determining the general characteristics of a particular implant device. However, present prior art systems for capturing such image data do not facilitate the accurate identification of the device properties, such as manufacturer, model, date, expiration, size, etc. The image data obtained may therefore also include indicia, such as markers, which may be arranged in a unique manner for identifying the device.

Therefore, a present need is felt to provide a system and method of rapidly determining the manufacturer and model and other information relating to an implanted medical device. The benefits, embodiments, and/or characterizations described herein are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below.

### SUMMARY OF THE INVENTION

The present invention is directed to a system and method for rapidly and accurately identifying an implantable medical device, said identification including, for example, the manufacturer and model or type of the implantable device, the date the implant was implanted, any recall or other future data associated with the implant since the date of implanting the device, and further assisting the surgeon and other medical professionals in identifying the instruments used to adjust and/or remove the medical device. One or more characteristics of the implant may be identified using the system and method of the present disclosure from an image of the implant or indicia readable on the implant and retrieved by various means of imaging an implantable device after it has been implanted.

The system and method described in the present disclosure utilizes, for example, x-ray data, magnetic resonance imaging (MRI) data or computed tomography (CT) data, which may be used to provide a surgeon or medical professional with accurate means of identifying implant device properties, such as manufacturer, model, date, expiration, size, etc. In one particular embodiment, the image data obtained may include indicia, such as markers, which may be arranged in a unique manner for identifying the device. In yet other embodiments, the image of the implant device may have other unique characteristics, such as geometric characteristics, that are associated with a particular implant.

Medical implants in this application include implants that are used to improve the health of the patient and also those used for aesthetic reasons. They may include spinal implants, cardiovascular implants, orthopedic implants, neurological implants or other medical apparatus inserted into or surgically attached to a patient, including screws, plates, rods, prosthetics, pacemakers, etc.

By way of example but not limitation, in total joint replacement, if the medical implant device is monitored for any change in alignment, wear or loosening during the lifetime of the implant, the system and method described herein may be used to identify the specific implant and the associated specifications for that implant, which over the course of several years and often required follow-up surgical procedures, may not be immediately discoverable from the patient's medical files and or known to the surgeon.

In further embodiments of the present disclosure, the medical implant device may be recognized by the placement and/or pattern of one or more radiolucent or other readable markers embedded within or adjacent to the surface of the implant device. In yet another embodiment, a plurality of markers may be arranged to form a readable bar code or other pattern-coded indicia. In one embodiment the markers may be formed from thin strips of material that have at least one surface comprising an adhesive element, which may be selectively adhered to the surface of the implant device by a manufacturer, distributor or user of the medical implant device.

An independent or networked computational piece of machinery, hereinafter referred to as an image-displaying apparatus, may be used for recognizing identifying marker(s) and comparing the identifying marker(s) to a database of known medical implant devices to determine the precise implant associated with the identifying marker(s).

According to one aspect of the present disclosure, a system for identifying a medical implant is disclosed, comprising:

an implanted device, the implanted device comprising one or more indicia;

a database containing a plurality of records for various implantable devices, each of the various implantable device associated with at least one record comprising associated indicia and other data relating to the implantable device;

an image-displaying apparatus comprising means for displaying a user interface, the image-displaying apparatus and user interface comprising means for accessing the database; wherein the one or more indicia are discernible by at least one of the following: an x-ray, fluoroscopy, computed tomography, electromagnetic radiation and magnetic resonance imaging;

wherein the one or more indicia are arranged in a manner that is unique to a particular implanted device; and

wherein the image-displaying apparatus and the user interface are configured to access the records in the database and compare the one or more discernible indicia to the plurality of records for identifying the other data relating to the implantable device.

The system may also employ means for ensuring confidentiality of the patient related information. As those skilled in the art will appreciate, any computers or computational machinery discussed herein may include an operating system (e.g., Windows 7, Windows Vista, NT, 95/98/2000, OS2; UNIX; Linux; Solaris; MacOS, Snow Leopard; etc.) as well as various conventional support software and drivers typically associated with computers. The computers or computational machinery may be in a mobile or personal or business environment with access to a network. In an exemplary embodiment, access is through the Internet through a commercially-available web-browser software package. These and other benefits of the present disclosure are described in greater detail in the Detailed Description.

The Summary of the Invention is neither intended nor should it be construed as being representative of the full extent and scope of the present disclosure. The present disclosure is set forth in various levels of detail in the Summary of the Invention as well as in the attached drawings and the Detailed Description of the Invention and no limitation as to the scope of the present disclosure is intended by either the inclusion or non-inclusion of elements, components, etc. in this Summary of the Invention. Additional aspects of the present disclosure will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below. However, the claims set forth herein below define the invention.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

In the drawings:

FIG. 1 is a schematic view of a system for identifying a medical implant according to an embodiment of the present disclosure;

FIG. 2 is another schematic view of the system according to an embodiment of the present disclosure;

FIG. 3 is another schematic view of the system according to an embodiment of the present disclosure;

FIG. 4 is a perspective view of a medical implant device and associated indicia according to a certain embodiment of the present disclosure; and

FIG. 4 is a flow chart diagram showing the steps of a method for identifying a medical implant according to an embodiment of the present disclosure.

### DETAILED DESCRIPTION OF THE INVENTION

The invention provides a mobile application for identifying a medical implant device in a human, or in certain embodiment an animal. Various aspects of the invention described herein may be applied to any of the particular applications set forth below or for any other types of user interfaces and image-displaying apparatus, or to various data management applications. The invention may be applied as a standalone system or method, or as part of an integrated package, such as a medical and/or data management application. It shall be understood that different aspects of the invention can be appreciated individually, collectively, or in combination with each other.

The system and method described in the present disclosure utilizes data, for example, x-ray data, magnetic resonance imaging (MRI) data or computed tomography (CT) data, which may provide a surgeon or medical professional with accurate means of identifying implant device properties, such as manufacturer, model, type, material, version, revision, date of manufacture, date of surgery, expiration, size, etc. In one particular embodiment, the data obtained may include indicia, such as markers, which may be arranged in a unique manner for identifying the device. In yet other embodiments, the image of the implant device may have other unique characteristics, such as geometric characteristics, that are associated with a particular implant.

In one particular embodiment of the present disclosure, the medical implant device may be recognized by the placement and/or pattern of one or more radiolucent or other readable markers embedded within or adjacent to the surface of the implant device. In yet another embodiment, a plurality of markers may be arranged to form a readable bar code or other pattern-coded indicia. An independent or networked computational piece of machinery or image-displaying apparatus may be used for recognizing identifying marker(s) and comparing the identifying marker(s) to a database of known medical implant devices to determine the precise implant associated with the identifying marker(s).

In one particular embodiment, the indicia are formed by the use of markers, which due to their location, orientation, sizing, spacing, etc. form a unique pattern. By way of example but not limitation, the markers may be arranged such that they form an array or pattern of dots or similar shapes when viewed by known imaging equipment, such as a CT or MRI scanner. The array or pattern may be unique to the particular implant, with certain dots or shaped markers corresponding to the manufacturer, while others refer to the date the implant was manufactured or the date the device was implanted. In yet another embodiment, the markers may be viewed in one particular orientation of the implant as a series of bars. In this embodiment, the spacing, height and thickness of the markers which form the bars may be unique to a particular implant, and provide a surgeon or other medical professional with a unique identification means for locating data files or records associated with the particular implant device. Further illustration of these concepts is provided in detail below.

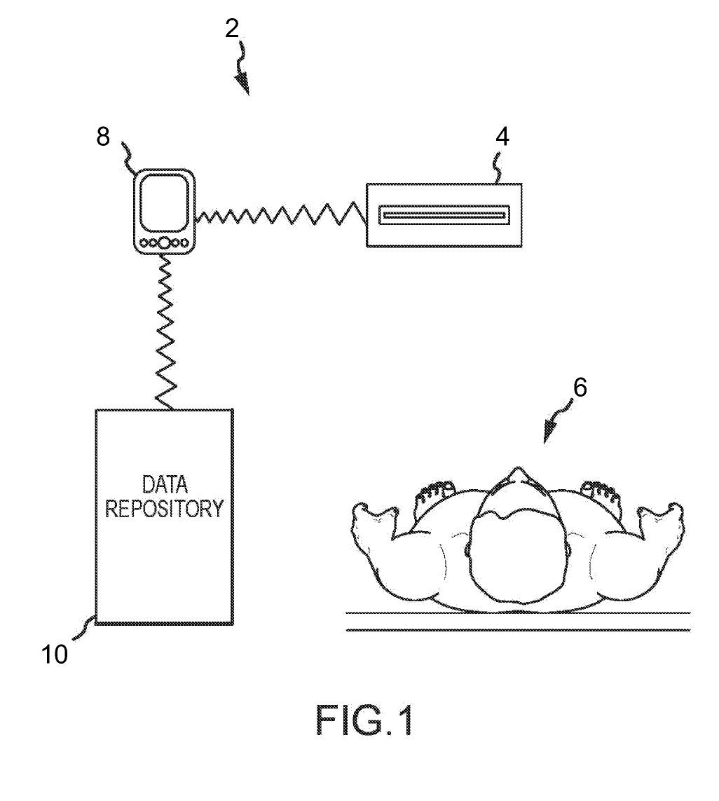

Referring now to the drawing figures, the appended FIG. 1 shows a system 2 for identification of medical implants. This embodiment of the present disclosure comprises a image-displaying device 8 and a scanning unit 4 for processing data related to at least one device implanted into the body of a patient 6. The scanning unit 4 and image-displaying device 8 are, according to a preferred embodiment, further associated with a central data repository 10. The scanning unit, which is used to convert one of several known image types into a format that can be viewed via the image-displaying device, may be of a number of different forms, so long as it is capable of obtaining image-related information relating to the device implanted into the patient.

The system and method shown in FIG. 1 is designed to be used with, in particular, an imaging examination device, e.g. an x-ray system, which may in certain embodiments be later digitized by employing the scanning unit 4 or other equipment available for such conversion. When the scanning unit 4 receives information on the implant from the x-ray system, for example, the data can be exported to the image-displaying device 8, which is connected in a preferred embodiment by wireless transmission to the central data repository 10.

In this embodiment, the central data repository 10 contains the data files or records associated with various manufacturers, models and types of implant devices. This system then compares the data exported to the mobile electronic device to the data files associated with various implant devices contained in the central data repository 10, e.g., the device characteristics, for accurately identifying the implant device.

In general, the scanning unit 4 provides optical pattern recognition of any two-dimensional or three-dimensional object, such as an x-ray image or other image. The scanning unit 4 may incorporate any algorithms, modalities, processes or formats known in the art without departing from the scope of the present disclosure. In one embodiment, the scanning unit 4 is used to digitally scan a two-dimensional image of a patient's anatomy and associated medical devices to be identified. In another embodiment, the scanning unit 4 may scan and process the data from the scan. For example, the comparison algorithm and methods for comparing two images described in U.S. Pat. No. 8,175,412, which is incorporated by reference herein in its entirety, may be used to compare the image obtained from the implanted device to a plurality of records stored in a central data repository to match the device to the data file associated with a device having the identical unique identification characteristics. Thus, according to this embodiment, the image-displaying device is not necessary.

Referring back to FIG. 1, the scanning unit converts the image of the implant device to a format that is received and is readable by the image-displaying device 8, which permits a user to call up the image of the implanted device and the associated data from the central data repository 10 in a convenient location. The image-displaying device 8 then communicates with and accesses data stored in the central data repository 10, which then communicates data associated with the medical device, such as the manufacture, model, type, revision, etc., back to the image-displaying device 8.

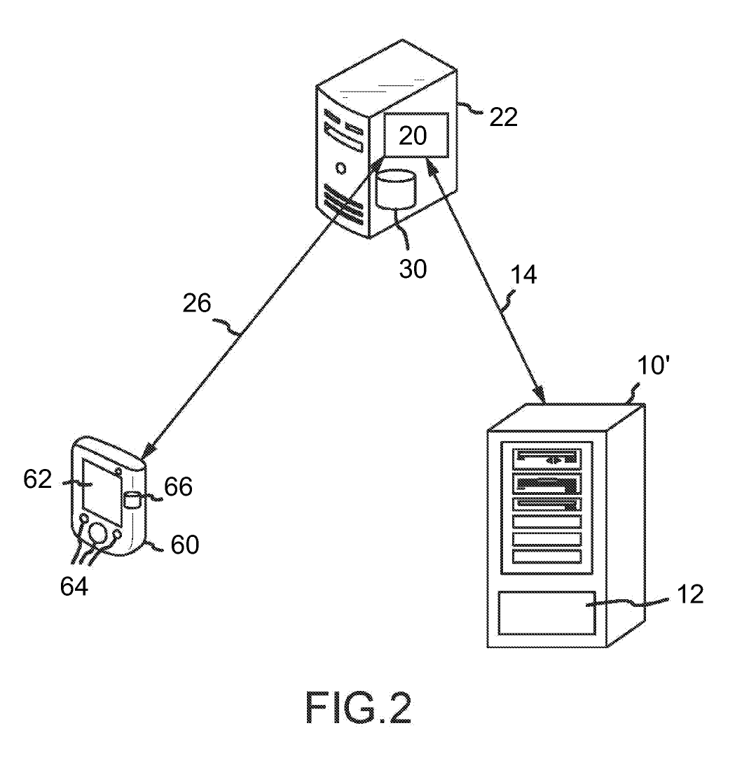

Referring now to the embodiment shown in FIG. 2, various types of mobile electronic devices may be used as the image-displaying device, which may also be connected to an information system located on a server 22 of the hospital. An electronic record of the patient can thus be automatically called up or created in which data, e.g. regarding the examination, treatment or condition of the patient are then stored. In this manner, the information system may be used to store new data related to the implant. This information may be transferred via https (Hyper Tech Transfer Protocol Secure) and/or SSL (Secure Socket Layer) protocols to ensure the transaction(s) and associated patient information exchanged between the user and the system described herein remain secure.

The central data repository associated with the system described herein may be utilized for storing data related to the transactions processed by a remote machine hosted application programming interface (API). The central data repository may have one or more permanent or removable memory storage devices, which may be periodically updated as new medical devices are introduced. Although the central data repository may be comprised of a single, integrated structure, it is expressly understood that several discrete storage mediums, which may or may not reside in the same location, may be used without departing from the spirit of the present disclosure.

One aspect of the present disclosure contemplates the use of various software elements to complete the transactions described above, provide the user with corresponding graphic user interface displays, etc. The software elements of the present disclosure may be implemented with any programming or scripting language such as C, C++, Java, COBOL, assembler, PERL, Visual Basic, SQL Stored Procedures, AJAX, extensible markup language (XML), with the various algorithms being implemented with any combination of data structures, objects, processes, routines or other programming elements. Further, it should be noted that the present disclosure of the claimed invention may employ any number of conventional techniques for data transmission, signaling, data processing, network control, and the like. Still further, embodiments of the claimed invention may detect or prevent breaches in security with a client-side scripting language, such as JavaScript, VBScript or the like.

A user interface provided in accordance with the invention described in varying embodiments herein may be displayed across a network such as the Internet or a wireless communication network. For example, an implementation may include a client device comprising a video display with at least one display page comprising data. The data may include medical data, such as patient data (e.g., medical history, patient history, prior medical treatment, physician entered data, etc.), surgical history and treatment logs (e.g., various visit or treatment information, patient assessments, patient notes, access history/usage), and any associated interfacing data (e.g., machine data or hospital-related data). In some embodiments, the data provided may be urgent data that may require immediate or quick actions in response.

Communication among the parties in accordance with the present disclosure may be accomplished through any suitable communication channels, such as, for example, a telephone network, an extranet, an intranet, Internet, point of interaction device (point of sale device, personal digital assistant, cellular phone, kiosk, etc.), online network communications, wireless communications, local area network (LAN), wide area network (WAN), networked or linked devices and/or the like. Moreover, although the network communications may be implemented with TCP/IP communications protocols, such communications may also be implemented using IPX, Appletalk, IP-6, NetBIOS, OSI or any number of existing or future protocols. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997), the contents of which are hereby incorporated by reference.

Another embodiment of the present disclosure is shown in FIG. 2, wherein a mobile device 60 is comprised of a display 62, one or more controls 64, and further comprises an internal memory 66 for storing data, such as image data and device characteristic data, for example. The mobile device 60 may communicate 26 with a server 22 or similar network device located at a hospital or other medical facility, which may house an information system 20 and, which may also have an internal memory 30 (and permits synchronization and communication with the mobile device 60). The server 22 communicates 14 with a central data repository 10′, where data relating to the dimensions, specifications, characteristics, appearances or other information or indicia related to specific medical devices may be stored and retrieved by the user of the mobile device 60.

The central data repository 10 according to this particular embodiment may also have one or more permanent or removable memory storage devices 12, which may be periodically updated as new medical devices are introduced. Although in FIG. 2 the central data repository 10 is shown as a single, integrated structure, it is expressly understood that several discrete storage mediums, which may or may not reside in the same location may be used without departing from the spirit of the present disclosure.

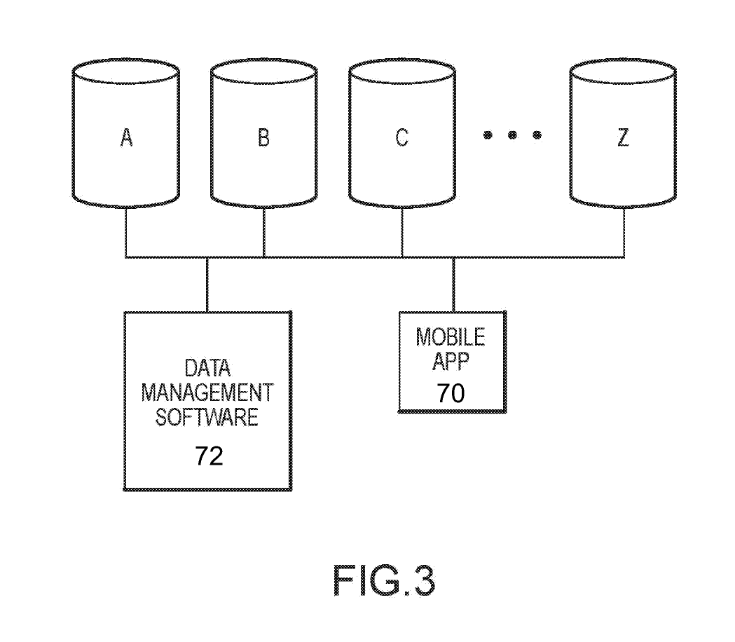

For example, referring now to FIG. 3, a schematic diagram is shown which permits a mobile device running a mobile application 70 to connect to a network that comprises a plurality of data storage mediums A, B, C, Z. The mobile application 70 may further communicate over this network with an algorithmic processor or data management software 72, which may reside, for example, on the server 22 (as shown in FIG. 2). According to yet another embodiment, the data management software 72 may reside on a remote server, and be accessed via login and password information supplied by the user of the mobile device 60 via the mobile application 70. In this embodiment, data related to a certain manufacturer's medical devices may reside in the storage medium A, while another medical device manufacture's data may reside on storage medium B, and so on. In this manner, the data management software 72 and/or the mobile application 70 may be configured to allow a user to access data on any one of these storage mediums shown in FIG. 3.

In one particular embodiment, indicia embedded within or adjacent to the surface of the device to be identified are arranged in a unique manner. It is expressly understood that the use of the term indicia is not intended to be limiting to writings or graphically represented indicia. To the contrary, the present disclosure involves in one embodiment the use of RFID elements or other embedded elements within the implant device, which may serve as indicia capable of translation to a user for identifying the device. In this particular embodiment, one or more unique RFID tags, each comprising a unique frequency or code (or in alternate embodiments, having the same frequency or codes), may be embedded in the medical implant device and may be detected by a reading device, such as the type described in U.S. Pat. No. 8,114,063, the entirety of which is incorporated by reference herein.

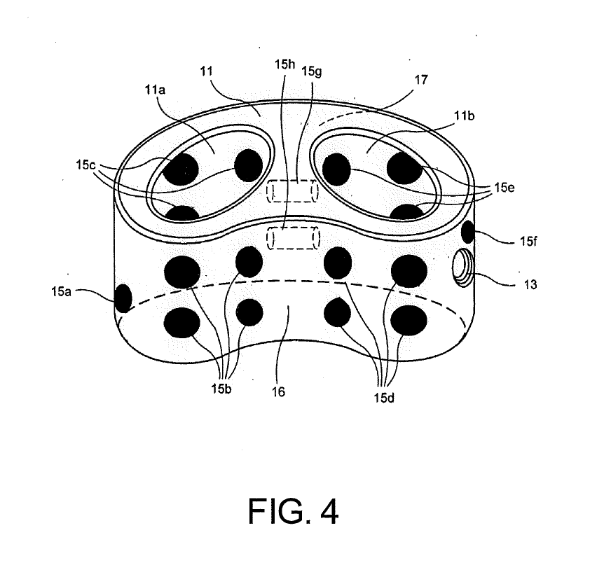

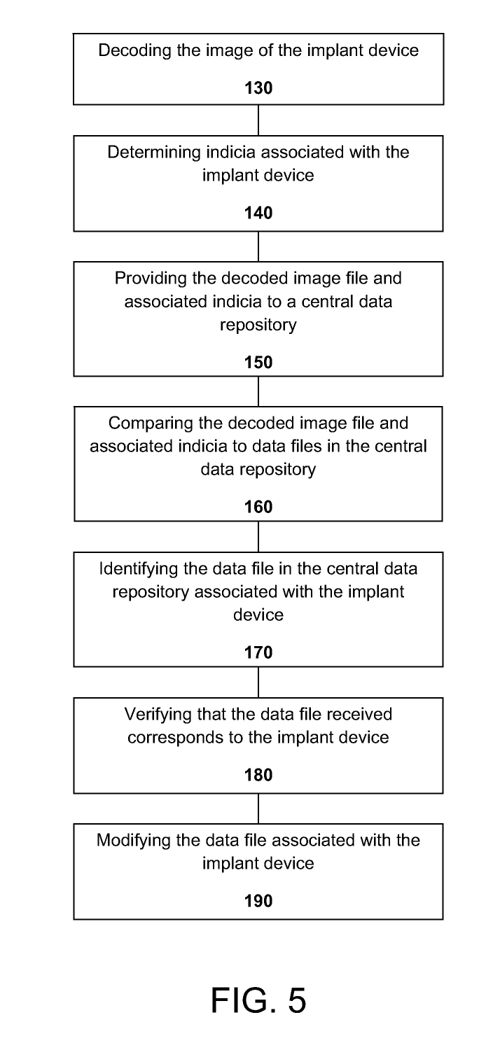

Referring now to FIG. 4, one particular embodiment of the present disclosure depicting an implantable device comprising markers for determining the unique identification characteristics of the device is shown. The implant device shown in FIG. 4 comprises a first surface 16 and a second surface 17 which are spaced apart from one another, thereby creating a void between the first surface 16 and second surface 17, wherein said void is accessible via apertures 11a, 11b shown in a top surface 11 of the implant. The implant may further comprise one or more attachment points 13 for attaching an instrument for inserting the implant device.

According to various embodiments described herein, the implant device may further comprise a series of markers, wherein the markers may be arranged in particular arrays (shown in FIGS. 4 as 15b, 15c, 15d and 15e). Markers may also be provided independently, such as in 15a and 15b shown in FIG. 4. According to one embodiment, a particular array of markers 15b may be comprised of different sized markers, different shaped markers and differently oriented markers to create a recognizable pattern or code by the particular size, shape, orientation, placement, etc. of the markers. By way of example but not limitation, the array of markers 15b may correspond to a particular manufacturer of the implant device. In this manner, a separate array of markers 15c may correspond to another characteristic of the implant device, such as the date of manufacture. Similarly, a separate array of markers 15d may correspond to a model or implant type, while yet another separate array of markers 15e may correspond to a date of surgery wherein the implant device shown in FIG. 4 was implanted.

In the embodiment shown in FIG. 4, the markers are shown as corresponding with dots, which may be embedded in the surface of the implant (e.g., implanted in the first surface 16 for arrays of markers 15b and 15d, and embedded into the second surface 17 for arrays of markers 15c and 15e). Alternatively, the markers may be placed on the surface of the implant, either by the manufacturer, distributor, surgeon or other medical professional.

In yet another embodiment, the markers may be comprised of rectangular or cylindrical prism shaped markers, which may be arranged in a horizontal, vertical or other orientation for representing characteristics of the implant device. As with the dot shaped marker shown in FIG. 4, the rectangular or cylindrical prism shaped markers may be arranged according to height, thickness, location and/or orientation, etc. to convey certain data relating to the medical device. The rectangular or cylindrical shaped markers may be embedded into the surfaces of the implant device or may be placed on the exterior or interior surfaces of the implant device as described above.

According to one embodiment, the implant device may further comprise one or more RFID elements (15g, 15h), which are ideally embedded into one or more of the surfaces of the implant device for further identifying the implant device or characteristics associated with the implant device. These RFID elements may be RFID tags, coils, transponders or other known RFID elements capable of being scanned and conveying data to a scanning unit external to the human or animal subject in which the implant device has been implanted into.

Referring now to FIG. 5, a preferred embodiment for performing a method of identifying a medical implant device is shown. In the first step 130, the implant device is imaged using one of a plurality of imaging techniques. Next, the image is decoded 140, either by removing unnecessary and/or unwanted data associated with the image or extracting one or more indicia associated with the implant device. The next step 150 involves providing the decoded image file or associated indicia to the central data repository, where the API or other equivalent software performs the algorithms necessary to compare the decoded image or associated indicia to a plurality of unique records of all known medical implant devices. Next, the unique medical implant device corresponding to the decoded image file or associated indicia is identified 160 and at least one data file associated with the identified implant device is provided to the user 170, preferably via a hand held device containing a user interface. In the next step 180, the user may visibly verify that the data file received corresponds to the implant device. The final step 190 is updating the patient records associated with the medical implant device.

Many of the components of the disclosure made herein may be described as being “in communication” or “in operable communication” with other components. Being “in communication” or “in operable communication” refers to any manner and/or way in which functional units or modules, such as, but not limited to, computers, laptop computers, tablets, PDAs, mobile networking devices, modules, network servers, routers, gateways, and other types of hardware and/or software, may be in communication with each other. Some non-limiting examples include: (i) activating or invoking another such functional unit or module, and (ii) sending, and/or receiving data or metadata via: a network, a wireless network, software, instructions, circuitry, phone lines, Internet lines, satellite signals, electric signals, optical signals, electrical and magnetic fields and/or pulses, and/or so forth.

Video displays may include devices upon which information may be displayed in a manner perceptible to a user, such as, for example, a computer monitor, cathode ray tube, liquid crystal display, light emitting diode display, touchpad or touch screen display, and/or other means known in the art for emitting a visually perceptible output. Video displays may be electronically connected to a client device according to hardware and software known in the art. Displays may be incorporated in one or more portable desktop accessories (“PDAs”) or other mobile devices, including but not limited to an iPhone.

At a client device, the display page may be interpreted by software residing on a memory of the client device, causing a file to be displayed on a video display in a manner perceivable by a user. The display pages described herein may be created using a software language known in the art such as, for example, the hypertext markup language (“HTML”), the dynamic hypertext markup language (“DHTML”), the extensible hypertext markup language (“XHTML”), the extensible markup language (“XML”), or another software language that may be used to create a computer file displayable on a video display in a manner perceivable by a user. Any computer readable media with logic, code, data, instructions, may be used to implement any software or steps or methodology. Where a network comprises the Internet, a display page may comprise a webpage of a type known in the art.

A display page according to the invention may include embedded functions comprising software programs stored on a memory, such as, for example, Cocoa, VBScript routines, JScript routines, JavaScript routines, Java applets, ActiveX components, ASP.NET, AJAX, Flash applets, Silverlight applets, or AIR routines.

A display page may comprise well known features of graphical user interface technology, such as, for example, frames, windows, tabs, scroll bars, buttons, icons, menus, fields, and hyperlinks, and well known features such as a touch screen interface. Pointing to and touching on a graphical user interface button, icon, menu option, or hyperlink also is known as “selecting” the button, icon, option, or hyperlink. Any other interface for interacting with a graphical user interface may be utilized. A display page according to the invention also may incorporate multimedia features.

A user interface may be displayed on a video display and/or display page. A server and/or client device may have access to data management and/or associated software. A user interface may be used to display or provide access to medical data. For example, a user interface may be provided for a web page or for an application. An application may be accessed remotely or locally. A user interface may be provided for a mobile application (e.g., iPhone application), gadget, widget, tool, plug-in, or any other type of object, application, or software.

Any of the client or server devices described herein may have tangible computer readable media with logic, code, or instructions for performing any actions described herein or running any algorithm. The devices with such computer readable media may be specially programmed to perform the actions dictated by the computer readable media. In some embodiments, the devices may be specially programmed to perform one or more tasks relating to data management. In some embodiments, the devices may communicate with or receive data collected from one or more measurement or sensing device, which may collect physiological data from a subject or from a sample collected from a subject.

In another embodiment, new or existing image management software could be incorporated with the system described above to analyze the two-dimensional image received from the scanning unit 4, and directly report the data associated with the medical device. For example, image management software known as DICOM could receive this type of data and be used in conjunction with the present disclosure.

In one alternative embodiment, the present disclosure may be used in conjunction with a plurality of identification markers. U.S. Pat. No. 7,901,945 is hereby incorporated by reference in its entirety for the purpose of supplementing this disclosure with respect to incorporating a method of image recognition by use of identification markers, biosensors, micro-fluidic arrays and optical character recognition.

According to yet another alternative embodiment, the scanning device 10 may be eliminated by incorporating the functionality of the scanning unit 4 with the mobile device 8. In this embodiment, the mobile device further comprises means for scanning the two-dimensional object and processing the data associated with the two-dimensional object without the need for a separate, independent scanning unit.

U.S. Pat. No. 7,855,812 is incorporated by reference herein in its entirety for the purpose of supplementing this disclosure with respect to scanning of high resolution two-dimensional images and converting those images for use in a mobile device such as a cellular phone.

The implants described herein may be made of a variety of different materials. These materials may include, by way of example but not limitation, stainless steel, titanium alloy, aluminum alloy, chromium alloy, and other metals or metal alloys. These materials may also include, for example, PEEK, carbon fiber, ABS plastic, polyurethane, resins, particularly fiber-encased resinous materials rubber, latex, synthetic rubber, synthetic materials, polymers, and natural materials. The markers described herein may similarly be made from a variety of materials, including but not limited to radiolucent materials.

While various embodiment of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims. For further illustration, the information and materials in appended Exhibit A hereto are expressly made a part of this disclosure and incorporated by reference herein in their entirety.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for identifying a device which has been implanted in a subject, comprising:
an implanted device, the implanted device comprising one or more indicia;
a database containing a plurality of records for various implantable devices, each of the various implantable device associated with at least one record comprising associated indicia and other data relating to the implantable device;
an image-displaying apparatus comprising means for displaying a user interface, the image-displaying apparatus and user interface comprising means for accessing the database; wherein the one or more indicia are discernible by at least one of the following: an x-ray, fluoroscopy, computed tomography, electromagnetic radiation and magnetic resonance imaging;
wherein the one or more indicia are arranged in a manner that is unique to a particular implanted device; and
wherein the image-displaying apparatus and the user interface are configured to access the records in the database and compare the one or more discernible indicia to the plurality of records for identifying the other data relating to the implantable device.

2. The system of claim 1, wherein the one or more indicia are comprised of one or more markers embedded within or adjacent to the surface of the implanted device.

3. The system of claim 2, wherein the one or more markers are formed from a radiolucent material.

4. The system of claim 3, wherein the one or more markers are sized to appear via an x-ray, fluoroscopy, computed tomography, electromagnetic radiation or magnetic resonance imaging as distinct from one another.

5. The system of claim 1, wherein the indicia are oriented to appear in a unique pattern of dots in at least orientation of the implanted device.

6. The system of claim 1, wherein the indicia are oriented to appear in a unique pattern of horizontal or vertical bars in at least orientation of the implanted device.

7. The system of claim 1, wherein the image-displaying apparatus is comprised of a portable desktop accessory and the user interface is comprised of a software application residing in memory on the portable desktop accessory.

8. The system of claim 1, wherein the image-displaying apparatus is comprised of a smart phone and the user interface is comprised of a software application residing in memory on the smart phone.

9. The system of claim 1, wherein the image-displaying apparatus is comprised of a personal computer and the user interface is comprised of a software application residing in memory on the personal computer.

10. The system of claim 9, wherein the database containing a plurality of records resides in memory on the personal computer.

11. The system of claim 1, wherein the database containing a plurality of records resides on a server accessible through a network architecture via at least one of a portable desktop accessory, a smart phone and a personal computer.

12. The system of claim 11, wherein the network architecture further comprises a wireless connection between the server and at least one of a portable desktop accessory, a smart phone and a personal computer.

13. A system for identifying a medical implant, comprising:
A medical implant comprising one or more indicia arranged in a manner that is unique to the medical implant;
a plurality of records associated with differing medical implants each of the plurality of records comprising data including one or more indicia and the manner in which they are arranged;
an image-displaying device comprising means for accessing the plurality of records;
wherein the one or more indicia are discernible by at least one of the following: an x-ray, fluoroscopy, computed tomography, electromagnetic radiation and magnetic resonance imaging; and
wherein the image-displaying apparatus and the user interface are configured to access the records in the database and compare the one or more discernible indicia to the plurality of records for identifying the other data relating to the implantable device.

14. The system of claim 13, wherein the one or more indicia are comprised of markers embedded within or adjacent to the surface of the medical implant.

15. The system of claim 14, wherein the markers are arranged as a series or matrix of dots such that the series of dots are discernible as a pattern when viewed via an x-ray, fluoroscopy, computed tomography, electromagnetic radiation of magnetic resonance imaging apparatus.

16. The system of claim 14, wherein the markers are arranged as a series of bars such that the series of bars are discernible as a pattern when viewed via an x-ray, fluoroscopy, computed tomography, electromagnetic radiation of magnetic resonance imaging apparatus.

17. The system of claim 14, wherein the markers are arranged as a series of lines such that the series of lines are discernible as a pattern when viewed via an x-ray, fluoroscopy, computed tomography, electromagnetic radiation of magnetic resonance imaging apparatus.

18. A method for identifying a medical implant that has been implanted in to human or animal subject, comprising the steps of:
decoding the image of the implant device;
determining indicia associated with the implant device;
providing the decoded image file and associated indicia to a central data repository;
comparing the decoded image file and associated indicia to data files in the central data repository;
identifying the data file in the central data repository associated with the implant device;
verifying that the data file received corresponds to the implant device; and
modifying the data file associated with the implant device.
